(19) United States
(12) Patent Application Publication
(10) Pub. No.: US 2026/0232345 A1
(43) Pub. Date: Aug. 13, 2026

(54) DILATING NEURO-MONITORING PROBE

(71) Applicant: Spineology Inc., St. Paul, MN (US)

(72) Inventors: Dan McPhillips, Ham Lake, MN (US); Joseph Gleason, Eagan, MN (US); Craig Bourgeault, St. Louis Park, MN (US); Garrett Ganske, Lino Lakes, MN (US)

(21) Appl. No.: 19/440,336

(22) Filed: Jan. 5, 2026

Related U.S. Application Data
(62) Division of application No. 16/877,087, filed on May 18, 2020, now abandoned.
(60) Provisional application No. 62/849,815, filed on May 17, 2019.

(51) Int. Cl. A61B 17/34; A61B 17/00

(52) U.S. Cl. CPC A61B 17/3417; A61B 17/00234; A61B 2017/00039; A61B 2017/00261; A61B 2017/3454

(57) ABSTRACT

A neuro-monitoring dilating probe can include a combined guide pin, dilator and neuro-monitoring probe all in one instrument. A distal end of the probe may include a conductive exposed tip and a tapered portion. The tapered portion can roll tissue out of the insertion path and allow for penetration into a disc annulus without an annulotomy. The tapered portion can be configured to reside fully in the annulus, ensuring no gap is formed between the probe and a subsequent dilator. Thus a nerve root will not become impinged in a gap. The proximal end of the probe may include a notch or other engagement feature to allow a tool to engage the probe to facilitate removal of the probe from the surgical site.

### PRIORITY

This application is a divisional of U.S. patent application Ser. No. 16/877,087, filed on May 18, 2020, which claims the benefit of U.S. Provisional Application Ser. No. 62/849,815, filed on May 17, 2019, which are hereby incorporated herein by reference in their entireties.

### FIELD

The present invention generally relates to surgical instruments. More particularly, the present invention relates to a dilating neuro-monitoring probe and access method.

### BACKGROUND

Implants for vertebral surgical procedures are commonly inserted into the vertebral space after a series of dilators, of increasing size, are used to create the path for the implant to travel through the patient's tissues. The larger the dilator, the more impingement and possible tissue damage that can occur. Thus, there is a continuing need to provide insertion tools and methods that minimize impingement and possible tissue damage.

### SUMMARY

Neural tissue cannot be observed with x-ray, making percutaneous pin and needle placement in the spine an unsettling experience. To aid surgeons in safely guiding the pins and/or needles that are so common in today's growing MIS spinal market, the present invention is a novel neuro-monitoring dilating probe system which enables the safe delivery of sharps down to the spinal surface and percutaneous access to the disc space for discectomy and/or implant insertion.

A neuro-monitoring dilating probe in one embodiment can include a combined guide pin, dilator and neuro-monitoring probe all in one instrument. A distal end of the probe may include a conductive exposed tip and a tapered portion. The tapered portion can roll tissue out of the insertion path and allows for penetration into a disc annulus without an annulotomy. The tapered portion can be configured to reside fully in the annulus, ensuring no gap is formed between the probe and a subsequent dilator. Thus a nerve root will not become impinged in a gap. The proximal end of the probe may include a notch or other engagement feature to allow a tool to engage the probe to facilitate removal of the probe from the surgical site.

The disclosure includes a neuro-monitoring dilating probe comprising a proximal end, an opposing distal end and an elongated shaft spanning between the proximal and distal ends. The distal end can terminate in a conductive exposed tip. The distal end can also define a tapered section that transitions from the elongated shaft to the conductive exposed tip. The tapered section can transition in a constant linear slope. The elongated shaft and the distal end can both be circular in cross section. The elongated shaft can have a constant diameter across its entire longitudinal length. The elongated shaft and the tapered section of the distal end can both be coated with a biocompatible electrically-insulating coating. The conductive exposed tip can terminate in a rounded end. The tapered section can have a longitudinal length of 10-20 mm. The proximal end can define a notch into a longitudinal surface thereof. The proximal end can also define an electrical connector.

The disclosure further includes a method of using a neuro-monitoring dilating probe. The method can include connecting the neuro-monitoring dilating probe to a stimulator source and inserting the neuro-monitoring dilating probe into a patient's tissues to neuro-map the patient's nerves to establish a safe surgical path and trajectory to a surgical site.

The method also can include inserting and advancing a neuro-monitoring dilating probe through a patient's tissues to map neural structures while providing electrical stimulus to a distal tip of the neuro-monitoring dilating probe. Then as the neuro-monitoring dilating probe is advanced, the patient's tissues are rolled out of a path of the advancement with a tapered portion of a distal end of the neuro-monitoring dilating probe to allow for penetration into a disc annulus without an annulotomy. The neuro-monitoring dilating probe is advanced until the tapered portion resides fully in the disc annulus.

Stimulus to the neuro-monitoring dilating probe can be ceased once the tip thereof is observed to be in the disc. A second dilator can be placed over the neuro-monitoring dilating probe and into the disc space such that no gap is formed between the neuro-monitoring dilating probe and the second dilator. The neuro-monitoring dilating probe can be removed after the step of placing the second dilator.

Discectomy surgical procedures are also provided herein.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a perspective view of a neuro-monitoring probe in accordance with embodiments of the invention.

FIG. 2 is an enlarged perspective view of the distal end of the neuro-monitoring probe of FIG. 1.

FIG. 3 is an enlarged perspective view of the proximal end of the neuro-monitoring probe of FIG. 1.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention.

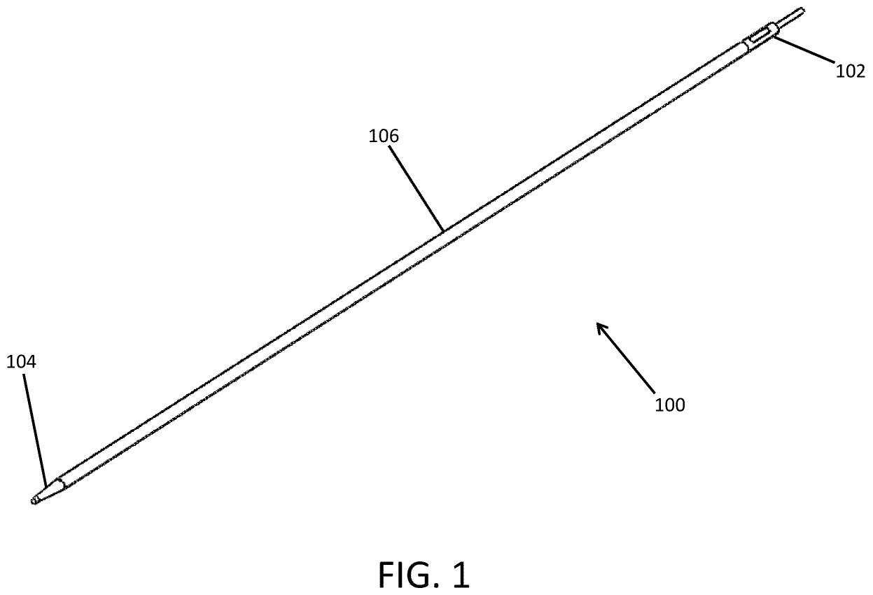

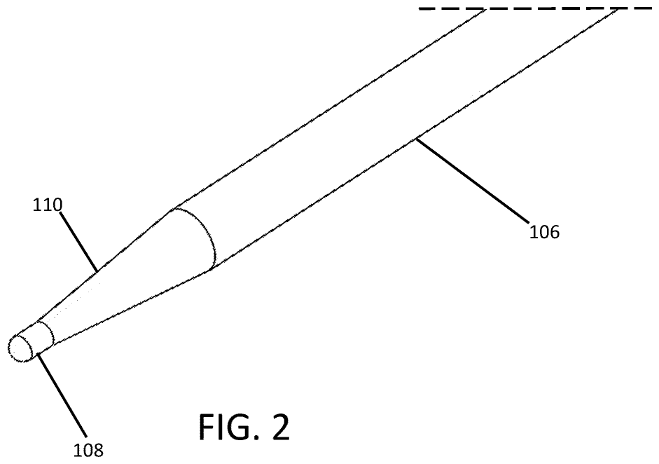

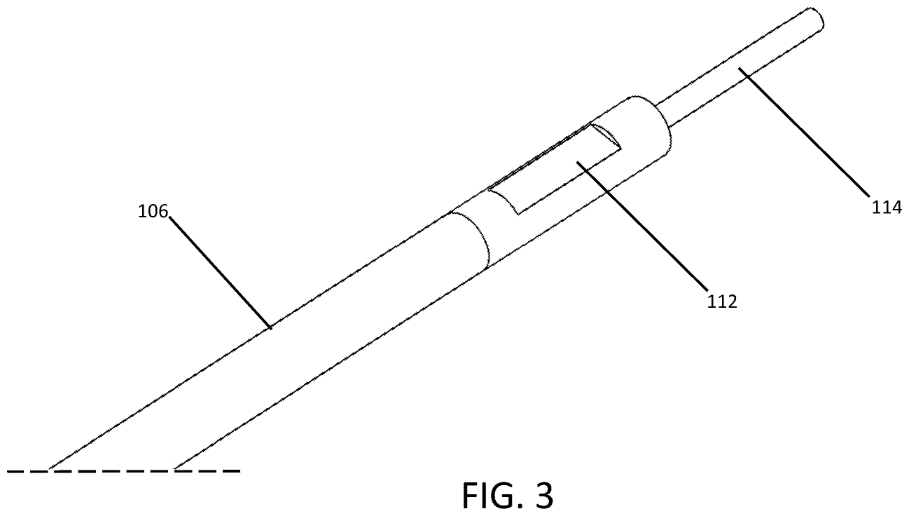

Referring to FIGS. 1-3, the neuro-monitoring dilating probe 100 comprises a combined guide pin, dilator and neuro-monitoring probe all in one instrument. Probe 100 may include a proximal end 102 and an opposing distal end 104 and elongated shaft 106 spanning between the proximal and distal ends.

Portions of the outer surface of the probe 100 may be coated with a biocompatible electrically-insulating coating, such as the elongated shaft 106 and the tapered section 110 of the distal end 104.

The distal end 104 may include a conductive exposed tip 108. The distal end 104 may define a tapered section 110 that transitions from the elongated shaft 106 to the tip 108 that has a narrower diameter than the shaft 106. The narrowed tip 108 and tapered section 110 roll tissue out of the insertion path to allow for penetration into a disc annulus without an annulotomy.

The tapered section 110 may generally be in the range of 10-20 mm in longitudinal length, such that the tapered section resides fully in the annulus, ensuring no gap is formed between the probe 100 and a subsequent dilator. Removing any gap also prevents a nerve root of the patient from becoming impinged in a gap.

The proximal end 102 may define a notch 112, slot, channel or other engagement feature that allows a tool to securely engage the probe 100 to facilitate removal of the probe 100 from the surgical site.

The proximal end 102 may also define an electrical connector 114 that can be coupled to an electrical lead, conduit, stimulator source, or instrument so that electrical signals can travel to/from the tip 108.

In use, after establishing the desired surgical trajectory, the neuro-monitoring dilating probe 100 may be connected to a stimulator source. The surgeon may then insert probe 100 to neuro-map the nerves to establish a safe surgical path and trajectory to the surgical site.

In a parapedicular approach the following steps may be taken: using frequent anteroposterior (AP) imaging, insert and advance probe 100 while targeting the superior lateral wall of the pedicle. As probe 100 is advanced, the electrified distal tip 108 will be seeking to evoke a neural response. If at any time a response is detected, the advancement is ceased and the probe 100 slightly retracted. Upon any trajectory change and re-advancement, if a response is again evoked, the probe 100 stimulus is titrated down in 1 mA increments to improve spatial sensitivity of the probe and then advancement can be continued. During the identification of an assumed safe trajectory, the probe 100 trajectory should be altered slightly in multiple directions around the original trajectory in order to evoke intermittent responses. This will aid in the mapping of the neural structures, while also insuring that the stimulus has not dropped below the threshold necessary to evoke a response.

When the probe tip 108 appears to be located on the superior lateral portion of the pedicle on an AP image, lateral imaging can be used to inspect the locations of the probe tip 108. The objective is to place tip 108 at the superior lateral edge of the pedicle, slightly posterior to the pedicle/vertebral body junction. If it is not at this location, the probe 100 can be retracted, the insertion angle altered, and then the probe re-advanced until the probe 100 is correctly positioned and no response is evoked.

In an embodiment of the present invention for transforaminal navigation for lumbar interdiscal procedures, the steps above are completed for the parapedicular approach, then the steps below are performed. Initially following the parapedicular approach steps will simplify placement of probe tip 108 in an intradiscal procedure by placing tip 108 medial to the exiting root prior to entering the inferior foramen.

With the probe tip 108 located on the superior lateral base of the pedicle, AP imaging is used to slide the tip 108 superiorly and medially along the base of the pedicle until the tip is located on the disc immediately superior to the coronal midpoint of the pedicle.

The probe tip 108 location is confirmed with lateral imagery. The exiting nerve root occupies the superior most portion of the foramen, making an approach to the disc through the inferior-most portion of the foramen desirable. It is not a requirement that probe 100 be centered on the disc, as slight variations in trajectory will enable access into the space when desired.

During this maneuver, if a neural response is evoked, it is helpful to determine which nerve root has responded. If the exiting root has been stimulated, probe tip 108 is likely too superior or lateral within the foramen. If the traversing root has been stimulated, the placement is too medial. Once it is determined which root has been stimulated, probe 100 is slightly retracted and repositioned further away from the offended root.

Once probe tip 108 is observed to be in the disc, the stimulus is turned to zero. The electrical wire is removed from the probe 100. A second dilator may then be placed over probe 100 and into the disc space. Probe 100 may then be removed. If desired, an access portal may then be placed over the second dilator to facilitate a discectomy. Once the access portal is placed, the second dilator may be removed and the discectomy may be performed.

In an embodiment of the present invention, an expandable intervertebral implant may be placed into the disc space through the access portal. In one aspect of such an embodiment, a trial may be used to determine the appropriate implant size.

In another embodiment, a guide wire or other place holder may be inserted into the disc space through the access portal. The access portal may then be removed and an insertion guide may be placed over such a place holder. An implant may then be placed using the insertion guide.

In yet another embodiment, an implant may be placed directly over a guide wire or through an access tube.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

1. A method of using a neuro-monitoring dilating probe, the method comprising:
connecting a neuro-monitoring dilating probe to a stimulator source;
inserting the neuro-monitoring dilating probe into a patient tissue bed that comprises nerves to neuro-map the nerves within the tissue bed to establish a safe surgical path and trajectory to a surgical site.

2. A method of using a neuro-monitoring dilating probe, the method comprising:
inserting and advancing the neuro-monitoring dilating probe through a patient's tissues to map neural structures while providing electrical stimulus to a distal tip of the neuro-monitoring dilating probe;
as the neuro-monitoring dilating probe is advanced, rolling the patient's tissues out of a path of the advancement with a tapered portion of a distal end of the neuro-monitoring dilating probe to allow for penetration into a disc annulus without an annulotomy; and
advancing the neuro-monitoring dilating probe until the tapered portion resides fully in the disc annulus.

3. The method of claim 2, further comprising ceasing stimulus to the neuro-monitoring dilating probe once the tip thereof is observed to be engaged with the disc annulus.

4. The method of claim 3, further comprising placing a second dilator over the neuro-monitoring dilating probe and into engagement with the disc annulus such that no gap is formed between the neuro-monitoring dilating probe and the second dilator.

5. The method of claim 4, further comprising removing the neuro-monitoring dilating probe after the step of placing the second dilator.

6. A method of advancing a probe through subject tissue toward a spinal disc of a subject, the method comprising:
(a) providing a neuro-monitoring dilating probe that extends lengthwise between a proximal end and a distal end, and wherein the neuro-monitoring dilating probe comprises an elongated shaft portion and a tapered section portion, wherein the elongated shaft portion extends lengthwise between proximal end and the tapered section portion, and the tapered section portion extends lengthwise from the distal end to the elongated shaft portion, wherein the tapered section portion transitions from the elongated shaft portion to an electrically conductive exposed tip disposed at the distal end;
(b) advancing the neuro-monitoring dilating probe into a tissue bed of the subject along a present trajectory toward a spinal disc of the subject;
(c) during the probe advancement along the present trajectory, applying an electrical stimulus to the electrically conductive exposed tip; and
(d) during the probe advancement along the present trajectory, monitoring the subject for a neural response to the applied electrical stimulus, wherein the monitoring will either indicate a detected neural response or indicate no detected neural response, wherein:
in response to the no detected neural response, continue the step of advancing the neuro-monitoring dilating probe into the tissue bed of the subject along the present trajectory toward the spinal disc of the subject; and
in response to the detected neural response, retracting the neuro-monitoring dilating probe a distance and subsequently advancing the neuro-monitoring dilating probe along an altered trajectory toward the spinal disc, wherein the altered trajectory is different from the present trajectory.

7. The method of claim 6, once the neuro-monitoring dilating probe is advanced along the altered trajectory toward the spinal disc, the altered trajectory becomes the present trajectory, and steps (b)-(d) are repeated.

8. The method of claim 7, wherein the application of the electrical stimulus to the electrically conductive exposed tip is terminated once the electrically conductive exposed tip is engaged with the spinal disc.

9. The method of claim 6, wherein the electrically conductive exposed tip is spherically shaped.

10. The method of claim 6, wherein the tapered section portion transitions from the elongated shaft portion to the electrically conductive exposed tip along a constant linear slope.

11. The method of claim 6, wherein the neuro-monitoring dilating probe has a lengthwise extending axis, and the elongated shaft portion and the tapered section portion both have a circular cross-section in planes perpendicular to the lengthwise extending axis.

12. The method of claim 6, wherein the neuro-monitoring dilating probe has a lengthwise extending axis, and the elongated shaft portion has a circular cross-section in planes perpendicular to the lengthwise extending axis, and the elongated shaft portion has a constant diameter along the lengthwise extending axis.

13. The method of claim 6, wherein the neuro-monitoring dilating probe has a lengthwise extending axis; and
wherein the elongated shaft portion has an exterior surface, and a notch disposed in the exterior surface proximate the proximal end.

14. The method of claim 13, wherein other than the notch, the elongated shaft portion has a circular cross-section in planes perpendicular to the lengthwise extending axis, and the elongated shaft portion has a constant diameter along the lengthwise extending axis.

15. The method of claim 6, wherein the neuro-monitoring dilating probe has a lengthwise extending axis; and
wherein the elongated shaft portion has an exterior surface; and
wherein the exterior surface of the elongated shaft portion is configured with an electrical insulation.

16. The method of claim 15, wherein the electrical insulation is a biocompatible electrically-insulating coating adhered to the exterior surface.

17. The method of claim 15, wherein other than the electrically conductive exposed tip, the tapered section portion (TSP) has a TSP exterior surface, and the TSP exterior surface is configured with the electrical insulation.

18. The method of claim 17, wherein the electrical insulation is a biocompatible electrically-insulating coating adhered to the exterior surface.

19. The method of claim 6, wherein the neuro-monitoring dilating probe comprises an electrical connector disposed at the proximal end.

20. The method of claim 19, wherein the neuro-monitoring dilating probe is configured to conduct the electrical stimulus from the electrical connector to the electrically conductive exposed tip.
